SEQUENCE LISTING (13 sequences)

SEQ ID NO: 1 - DNA, 2333 bp, Homo sapiens
ggcacgaggctagagcgatgccgggccggagttgcgtcgccttagtcctcctggctgccgccgtcagctgtgccgtcgcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccaatgctaaaggttatagtgaattctctgaagaatatgataaatacattcgtgccaagtggaaaagttatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgccatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtgagcacacgtctggcctacagaatgcggcctctgagacatgaagacaccatctccatgtgaccgaacactgcagctgtctgaccttccaaagactaagactcgcggcaggttctctttgagtcaatagcttgtcttcgtccatctgttgacaaatgacagatctttttttttttccccctatcagttgatttttcttatttacagataacttctttaggggaagtaaaacagtcatctagaattcactgagttttgtttcactttgacatttggggatctggtgggcagtcgaaccatggtgaactccacctccgtggaataaatggagattcagcgtgggtgttgaatccagcacgtctgtgtgagtaacgggacagtaaacactccacattcttcagtttttcacttctacctacatatttgtatgtttttctgtataacagccttttccttctggttctaactgctgttaaaattaatatatcattatctttgctgttattgacagcgatattattttattacatatcattagagggatgagacagacattcacctgtatatttcttttaatgggcacaaaatgggcccttgcctctaaatagcactttttggggttcaagaagtaatcagtatgcaaagcaatcttttatacaataattgaagtgttccctttttcataattactctacttcccagtaaccctaaggaagttgctaacttaaaaaactgcatcccacgttctgttaatttagtaaataaacaagtcaaagacttgtggaaaataggaagtgaacccatattttaaattctcataagtagcattgatgtaataaacaggtttttagtttgttcttcagattgatagggagttttaaagaaattttagtagttactaaaattatgttactgtatttttcagaaatcaaactgcttatgaaaagtactaatagaacttgttaacctttctaaccttcacgattaactgtgaaatgtacgtcatttgtgcaagaccgtttgtccacttcattttgtataatcacagttgtgttcctgacactcaataaacagtcactggaaagagtgccagtcagcagtcatgcacgctgataaaaaaaaaaaaaaaaaa

SEQ ID NO: 2 - protein, 395 aa, Homo sapiens
MPGRSCVALVLLAAAVSCAVAQHAPPWTEDCRKSTYPPSGPTYRGAVPWYTINLDLPPYKRWHELMLDKAPMLKVIVNSLKNMINTFVPSGKVMQVVDEKLPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLTLNERFSINGGYLGILEWILGKKDAMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW

SEQ ID NO: 3 - protein, 26 aa, Homo sapiens
ASSFAGYVGMLTGFKPGLFSLTLNER

SEQ ID NO: 4 - protein, 11 aa, Homo sapiens
ESLDVYELDAK

SEQ ID NO: 5 - protein, 22 aa, Homo sapiens
ILAPAYFILGGNQSGEGCVITR

SEQ ID NO: 6 - protein, 11 aa, Homo sapiens
KDAMWIGFLTR

SEQ ID NO: 7 - protein, 17 aa, Homo sapiens
LPGLLGNFPGPFEEEMK

SEQ ID NO: 8 - protein, 20 aa, Homo sapiens
LTVYTTLIDVTKGQFETYLR

SEQ ID NO: 9 - protein, 27 aa, Homo sapiens
STYPPSGPTYRGAVPWYTINLDLPPYK

SEQ ID NO: 10 - protein, 43 aa, Homo sapiens
TSQENISFETMYDVLSTKPVLNKLTVYTTLIDVTKGQFETYLR

SEQ ID NO: 11 - protein, 7 aa, Homo sapiens
VIVNSLK

SEQ ID NO: 12 - protein, 8 aa, Homo sapiens
WHELMLDK

SEQ ID NO: 13 - protein, 20 aa, Homo sapiens
WYVVQTNYDRWKHPFFLDDR

(19) United States
(12) Patent Application Publication
(10) Pub. No.: US 2011/0091439 A1
(43) Pub. Date: Apr. 21, 2011

(54) COSMETIC USE OF ACID CERAMIDASE TYPE PROTEINS

(75) Inventors: Dominique Bernard, Paris (FR); Lucie Simonetti, Vincennes (FR); Isabelle Castiel, Nice (FR)

(73) Assignee: L'OREAL, Paris (FR)

(21) Appl. No.: 12/809,428

(22) PCT Filed: Dec. 18, 2008
(86) PCT No.: PCT/IB2008/055423

§ 371 Date: Oct. 25, 2010

Related U.S. Application Data
(60) Provisional application No. 61/020,613, filed on Jan. 11, 2008.

(30) Foreign Application Priority Data
Dec. 18, 2007 (FR) 0759929

(51) Int. Cl. A61K 38/46; A61K 38/00; G01N 33/53; C12Q 1/68; C12N 5/071; A61Q 19/00; A61Q 19/08

(52) U.S. Cl. 424/94.6; 514/18.8; 435/7.21; 435/6; 435/325

(57) ABSTRACT

A method for preventing and/or treating the signs of skin aging comprises administering as an agent into an individual in need thereof an effective amount of acid ceramidase, polypeptides derived from this protein or analogues thereof, a nucleic sequence encoding such a polypeptide, or an agent for modulating the expression or the biological or biochemical activity of such a polypeptide. A methods for characterizing a state of an epithelium comprises determining a content of acid ceramidase, polypeptides derived from this protein or analogues thereof, or a nucleic sequence encoding such a polypeptide in an epidermis sample.

### BACKGROUND

Embodiments of the present invention relate to methods for preventing and/or treating the signs of skin aging by administering as an agent into an individual in need thereof an effective amount of acid ceramidase, polypeptides derived from this protein, for example derived from the proteolysis thereof, or analogues thereof, a nucleic sequence encoding such a polypeptide or an agent for modulating the expression or the biological or biochemical activity of such a polypeptide.

Embodiments also relate to a method for characterizing a state of an epithelium, and in particular an epidermis, by determining a content of acid ceramidase, polypeptides derived from this protein or of analogues thereof, or a nucleic sequence encoding such a polypeptide.

Epithelia are tissues of which the cells are joined to and interlinked with one another and lie on a basal membrane. They form either an external covering, for example at the surface of the skin, or the epidermis, or an internal covering, at the surface of a mucosa. They can also form glands.

More specifically, these epithelia are structures of which the homeostasis results from the use of a finely regulated set of intracellular and extracellular signals acting at all the stages of cell proliferation, migration and differentiation, and also of the synthesis of the various extracellular matrix components. These signals can in particular result from the action of factors produced by keratinocytes.

The maintaining of the correct physiological functions of an epithelium involves in particular epithelial terminal differentiation and/or proteoglycan synthesis.

With respect to the epidermis, it is an epithelium, conventionally divided up into a basal layer of keratinocytes containing, in particular, skin stem cells and constituting the germinative layer of the epidermis, a “spiny” layer constituted of several layers of polyhedral cells placed on the basal layer, a “granular” layer comprising one to three layers said to be of flattened cells containing distinct cytoplasmic inclusions, keratohyalin granules, and finally, a set of upper layers, called horny layer (or stratum corneum) constituted of keratinocytes at the terminal stage of their differentiation, called corneocytes.

The stratum corneum, the outermost part of the skin, which performs the function of a barrier between the organism and the environment, and the hair shaft, the emerging part of the hair follicle which constitutes the head of hair, both represent the result of the keratinocyte differentiation process. Epidermal differentiation follows a process of maturation in which keratinocytes from the basal layer differentiate and migrate so as to result in the formation of corneocytes, which are completely keratinized dead cells. This differentiation is the result of perfectly coordinated phenomena that will result in the thickness of the epidermis being kept constant and, thus, ensure the homeostasis of the epidermis.

Many skin disorders or pathologies can result from a dysfunction of epidermal homeostasis.

For example, in the case of aged skin, this dysfunction is generally manifested through the appearance of wrinkles (microrelief and deep wrinkles), a loss of elasticity, a rough feel and dryness. From the histological point of view, a flattening of the dermo-epidermal junction and a decrease in thickness of the dermis and of the epidermis are observed. The collagen and glycosaminoglycan content decreases. The barrier function of the skin is impaired. All these phenomena are increased by chronic exposure to the sun.

Similarly, this dysfunction may be worsened in women, during the menopause.

Embodiments of the present invention result from, for example, the characterization, by the inventors, of the expression of the acid ceramidase protein in the stratum corneum of the human epidermis, such as dry and aged human epidermis.

Acid ceramidase (also known as N-acylsphingosine amidohydrolase) is a heterodimeric protein, the precursor form of which comprises 395 amino acids (SEQ ID NO 2) and has a molecular weight of approximately 55 kDa. The mature form is obtained by cleavage of the precursor form and comprises a nonglycosylated alpha-subunit and a glycosylated beta-subunit. The et-subunit has a molecular weight of approximately 13 kDa and the β-subunit has a molecular weight of approximately 40 kDa.

Post-translational modifications, such as N-acetylglycosylation, are capable of modifying its activity. Acid ceramidase is a lysosomal enzyme of the hydrolase type, responsible for the hydrolysis of ceramide to sphingosine and to fatty acids.

A deficiency in the activity of this enzyme is reflected by an accumulation of ceramides in the lysosomes, and appears to be involved in Farber's disease.

Acid ceramidase is expressed in many tissues, including the heart and the epidermis. It has recently been observed that acid ceramidase has an activity in keratinocyte differentiation (Houben et al., J Lipid Res, 2006, 47:1063-70).

Jin et al. (Acta Derm Venereol., 1994, September; 74(5):337-40) have observed an increase in a ceramidase type activity in aged skin.

On the other hand, to the inventors' knowledge, acid ceramidase had not up until now been identified as being a protein of which the expression is decreased in the aged and dry human stratum carneum.

Thus, against all expectations, acid ceramidase is found also to be a potential marker for the physiological state of the skin, such as in terms of aging. In fact, this disclosure demonstrates a significant and unexpected decrease in acid ceramidase expression during aging of the epidermis.

### SUMMARY

In embodiments, a subject of the present invention is a cosmetic or alternatively nontherapeutic method for preventing and/or treating the signs of skin aging comprising administering as an agent into an individual in need thereof an effective amount of at least one polypeptide derived from acid ceramidase and, for example, having an amino acid sequence encoded by a nucleic acid sequence represented entirely or partly by a sequence represented by SEQ ID NO 1, an analogue thereof or a fragment thereof, at least one nucleic sequence encoding such a polypeptide or at least one agent for modulating the expression or the biological or biochemical activity of such a polypeptide.

In embodiments, a subject of the present invention is a method for preparing a composition, such as a therapeutic composition, for preventing and/or treating the signs of skin aging comprising an effective amount of at least one polypeptide derived from acid ceramidase and, for example, having an amino acid sequence encoded by a nucleic acid sequence represented entirely or partly by a sequence represented by SEQ ID NO 1, an analogue thereof or a fragment thereof, at least one nucleic sequence encoding such a polypeptide or at least one agent for modulating the expression or the biological or biochemical activity of such a polypeptide.

### DETAILED DESCRIPTION OF EMBODIMENTS

The term “signs of skin aging” is intended to mean all the modifications of the external appearance of the skin due to aging, whether it is chronological and/or photoinduced, for instance wrinkles and fine lines, wizened skin, lack of elasticity and/or of tonicity of the skin, thinning of the dermis and/or degradation of the collagen fibers, thereby leading to the appearance of soft and wrinkled skin.

For the purpose of the present invention, the expression “effective amount” is intended to denote the minimum amount required for the observation of the expected effect, namely a cosmetic effect or a therapeutic effect, it being understood that the effective amounts required for obtaining a cosmetic effect or a therapeutic effect may, as appropriate, be identical or different.

For the purpose of the invention, the term “cosmetic method” is intended to denote a method to provide an esthetic effect and/or an effect of comfort.

For the purpose of the invention, the term “therapeutic composition” is intended to denote a composition to provide a prophylactic or curative effect with respect to epithelial, such as epidermal, disorders recognized as reflecting a pathological state.

For the purpose of the invention, the term “prophylactic” or “preventive” is intended to mean a decreased risk of occurrence of a phenomenon, for example a pathology.

A composition in accordance with the invention may be for preventing and/or treating thinning of an epidermis and/or a loss of firmness, of elasticity, of density and/or of tonicity of an epidermis and/or the formation of wrinkles and fine lines.

According to another embodiment, a composition in accordance with the invention may be for preventing and/or treating signs of dryness of the skin, such as for preventing and/or treating dehydration of an epithelium, such as of an epidermis.

A composition in accordance with the invention may in particular be for preventing and/or treating the effects of chronological aging of an epithelium, such as of an epidermis or of the lips or of the scalp.

According to another aspect, the present invention also relates to a method for screening for biological or chemical compounds for modulating, such as activating, the expression and/or the biological or biochemical activity of said polypeptide by administering as an agent into an individual in need thereof an effective amount of at least one polypeptide in accordance with the invention.

In embodiments, the present invention relates to a method for screening for anti-aging active agents, comprising at least the steps consisting in:

a) bringing at least one cell type capable of expressing a polypeptide in accordance with the invention, such as acid ceramidase or a derivative thereof, into contact with at least one chemical or biological test compound, under conditions suitable for manifestation of the expression of said polypeptide, and

b) determining the content of said polypeptide.

According to yet another of its aspects, the present invention also relates to a method for characterizing, in vitro or ex vivo, a state of an epithelium, such as an epidermis, by administering as an agent into an individual in need thereof an effective amount of at least one polypeptide in accordance with the invention, or of at least one nucleic acid sequence encoding said polypeptide.

According to another of its aspects, the present invention relates to a noninvasive, such as cosmetic, method for characterizing the surface state of an epithelium, such as an epidermis, comprising at least the qualitative or quantitative characterization of the expression and/or of the biological or biochemical activity of a polypeptide in accordance with the invention, such as acid ceramidase, or of a derivative or fragment thereof.

According to a variant embodiment, the datum or value obtained may be assessed in comparison to a reference datum or value, obtained for example from at least one epithelium, such as one epidermis, which is different from that which is the subject of the characterization, and the state of which is known.

According to another of its aspects, the present invention is also directed toward a noninvasive, such as cosmetic, method for characterizing the effectiveness of a cosmetic or therapeutic treatment aiming to compensate for the signs of skin aging, comprising at least the qualitative or quantitative characterization of the expression and/or of the biological or biochemical activity of a polypeptide in accordance with the invention, such as acid ceramidase, or of a derivative or fragment thereof.

According to a variant embodiment, the datum obtained at the end of the characterization may also be examined in comparison to a reference value or datum. This reference value or datum may be a datum obtained from the epithelium, such as from the epidermis, that is to be subjected to the treatment, prior to the administration of said treatment or within a shorter chronological time in relation to the treatment start date.

As demonstrated herewith by the disclosure, the methods according to the invention are particularly advantageous since their implementation does not require an invasive procedure.

The methods of the invention may be carried out in vitro, ex vivo or in vivo.

Indeed, the localization of the new biomarker for aging, such as acid ceramidase, in the stratum corneum makes a quantitative or qualitative characterization of the expression of this protein possible by mere topical sampling. The sampling method may, for example, be a stripping technique comprising applying, to the epithelium under consideration, such as an epidermis, a portion of adhesive tape. On detaching this adhesive tape, a fraction of the epithelium, for example an epidermal fraction, is removed. After protein extraction, said fraction is then analyzed by conventional methods, such as immunoenzymatic assay or Western-blot analysis.

### Polypeptide Definition

According to one embodiment, a polypeptide suitable for the invention may have an amino acid sequence represented entirely or partly by a sequence represented by SEQ ID NO 2, or an analogue thereof, or a fragment thereof.

For the purpose of the present invention, the term “acid ceramidase” is intended to denote, in general, unless otherwise indicated, the sequence (SEQ ID NO 2) of the protein having or not having undergone post-translational modifications, of the N-acetylglycosylation type, on the asparagine residues at position 173, 195, 259, 286, 342 or 348, capable of modifying its apparent molecular weight or its isoelectric point.

The primary sequence of a polypeptide, i.e. the succession of the amino acids, may determine sites specifically recognized by protease-type enzymes, such as trypsin, which, once the recognition of these sites has become effective, will induce cleavage of the polypeptide by proteolysis. This proteolysis results in the generation of various peptides, or proteolytic fragments, of the acid ceramidase.

The inventors have detected the presence of such peptides in the stratum corneum.

Consequently, the invention also extends to the proteolytic fragments of acid ceramidase.

Thus, according to one particular embodiment, a polypeptide suitable for the invention may have an amino acid sequence chosen from SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12 and SEQ ID NO 13, and mixtures thereof.

The term “analogue of a polypeptide” is intended to denote any polypeptide exhibiting a sequence homology, for example, with respect to one of the characteristic sequences of said polypeptide, and also a biological or biochemical activity of the same nature.

This compound may be a peptidomimetic agent.

The homology may be at least 85%, for example at least 90%, and for example at least 95%. The homology may be determined by visual comparison or by means of any computer tool generally used in the field, such as the BLAST programs available on www.ncbi.nlm.nih.gov and used with the default parameters.

The sequence homology may result from modifications derived from mutation or variation in the sequences of the peptides according to the invention, originating either from the deletion or from the insertion of one or more amino acids, or else from the substitution of one or more amino acids in the characteristic sequences of a polypeptide according to the invention.

For the purpose of the invention, the term “polypeptide fragment” is intended to denote any portion of a polypeptide in accordance with the invention comprising at least 4, at least 6, at least 8, or at least 12 consecutive amino acids of said polypeptide, and a substantially similar biological or biochemical activity.

The teen “characteristic sequence of the polypeptide” is, from the viewpoint of acid ceramidase, intended to be directed to the sequence represented by SEQ ID NO 2.

In general, the polypeptide analogues may comprise conservative substitutions relative to the amino acid sequence of the natural polypeptide.

Several of these modifications may be combined.

By way of example of mutations that may be considered in the present invention, mention may be made, nonexhaustively, of the replacement of one or more amino acid residues with amino acid residues having a similar hydropathic index, without however substantially affecting the biological properties of the polypeptide, for example, its enzymatic activity.

The hydropathic index is an index assigned to amino acids as a function of their hydrophobicity and of their charge (Kyte et al. (1982), J. Mol. Biol., 157: 105).

A polypeptide or analogue that is also covered by the present invention may be a polypeptide having undergone one or more post-translational modification(s).

The term “post-translational modification(s)” is intended to encompass all the modifications that a peptide or a protein is capable of undergoing at the end of its synthesis in a cell, such as, for example, one or more phosphorylation(s), one or more thiolation(s), one or more acetylation(s), one or more glycosylation(s), one or more lipidation(s), such as a farnesylation or a palmitoylation, a structural rearrangement such as the formation of disulfide bridges and/or cleavage within the peptide sequence.

The analogue has, moreover, substantially the same biological or biochemical activity as the natural polypeptide.

According to one embodiment, a polypeptide suitable for the implementation of the invention may also be a natural or synthetic polypeptide, as appropriate, capable of being obtained after enzymatic or chemical lysis of acid ceramidase, or by chemical or biological synthesis, or by extraction from a biological tissue, for instance the skin, expressing this polypeptide naturally or after transfection thereof, and also the various post-translational forms of said polypeptide, or else any natural or synthetic polypeptide of which the sequence completely or partially (entirely or partly) comprises an amino acid sequence mentioned above, for example the variants and the analogues.

Those skilled in the art can obtain a polypeptide in accordance with the invention by means of recombinant DNA-base methods, for instance those described in the manual “Molecular Cloning—A Laboratory Manual” (2^{nd} edition), Sambrook et al., 1989, Vol. I-III, Coldspring Harbor Laboratory, Coldspring Harbor Press, NY, (Sambrook).

According to another embodiment, a polypeptide suitable for the implementation of the invention may also be a polypeptide as defined above, in which at least one residue has been replaced with an amino acid residue having a similar hydropathic index, as defined above.

According to another embodiment, a polypeptide suitable for the implementation of the invention may also be a polypeptide as defined above, fused with another polypeptide, a hydrophilic or hydrophobic targeting agent, a bioconversion precursor, or a luminescent, radioactive or colorimetric labeling agent.

In a nonlimiting manner, mention may be made, as an example of compounds that can be coupled with a polypeptide in accordance with the invention, of fluorescent proteins such as Green Fluorescent Protein, fluorescent chemical compounds such as rhodamine, fluorescein or Texas Red®, phosphorescent compounds, radioactive elements, such as ³H, ¹⁴C, ₃₅S, ¹²¹I or ¹²⁵I, or colorimetric labeling agents such as chromogenic substrates sensitive to the action of galactosidase, of peroxidase, of chloramphenicol acetyltransferase, of luciferase or of alkaline phosphatase.

Depending on the nature of the compounds that can be coupled with a polypeptide in accordance with the invention, the coupling may be performed by chemical methods, for example, by means of reactive chemical functions, or by molecular biology methods known to those skilled in the art.

### Definition of Nucleic Acid Sequences

According to one embodiment, the present invention also relates to nucleic acid sequences encoding a polypeptide of the invention and to the employment thereof in the various uses and methods in accordance with the invention.

Thus, the present invention also relates to the use of nucleic acid, such as deoxyribonucleic acid or ribonucleic acid, sequences encoding a polypeptide in accordance with the invention, such as the sequences corresponding at least to a nucleic acid sequence represented by SEQ ID NO 1, analogues thereof or a fragment thereof, for the preparation of a composition in accordance with the invention.

For the purpose of the present invention, the term “nucleic acid sequence fragment” is intended to denote a nucleic acid sequence encoding all or part of a polypeptide in accordance with the invention, or an analogue of said polypeptide, such as a nucleic acid sequence represented by SEQ ID NO 1 or an analogue thereof.

The expression “analogue of a nucleic acid sequence” is intended to denote any nucleic acid sequence possibly resulting from the degeneracy of the nucleic acid code, and encoding a polypeptide with a sequence identical or analogous to that of the polypeptide encoded by said nucleic acid sequence.

The nucleic acid sequences may be derived from all possible origins, i.e. either of animal, such as mammalian, such as human, origin, or of plant origin, or of microbial origin (viruses, phages, bacteria, inter alia) or else of fungal origin, without prejudice regarding whether or not they are naturally present in said organism of origin.

In the case in point, the invention also relates to the step of administering as an agent into an individual in need thereof an effective amount of isolated and purified nucleic acid fragments encoding the polypeptides considered according to the invention.

A nucleic acid sequence in accordance with the invention may comprise a sense, antisense or interfering sequence corresponding to a sequence encoding a polypeptide in accordance with the invention.

Thus, the present invention also relates to the use of nucleic acid, such as deoxyribonucleic acid or ribonucleic acid, sequences encoding a polypeptide in accordance with the invention.

The nucleic acid sequences according to the invention may, for example, be used for preparing the corresponding sense or antisense ribonucleic acid sequences.

A subject of the invention is also the use of any polynucleotide, having a ribonucleic or deoxyribonucleic acid sequence, comprising a sense or antisense sequence, such as small interfering RNA (siRNA), corresponding at least to the nucleic acid sequence SEQ ID NO 1 or an analogue thereof.

### Modulating Agent

According to another embodiment, the invention relates to the step of administering as an agent into an individual in need thereof an effective amount of an agent for modulating the expression and the activity of a polypeptide in accordance with the invention.

For the purpose of the invention, the term “modulate” is intended to mean, in relation to a given effect, the action of stimulating or inhibiting this effect.

The expression “agent for modulating acid ceramidase” is intended to mean a product capable of interfering with the activity, such as enzymatic activity, thereof.

The modulator may interfere indirectly by modifying the amount of endogenous enzyme produced, such as by promoting or, conversely, by decreasing the gene expression thereof and/or the translation thereof. Thus, any agent for modulating the gene or protein expression of acid ceramidase may be suitable for the invention.

The modulating agent may also interfere directly with the enzyme reaction catalyzed by acid ceramidase. It is, for example, an enzyme activator or inhibitor.

The term “biochemical activity” is intended to denote the enzymatic activity of acid ceramidase, such as its ceramide-hydrolyzing activity.

The term “biological activity” is intended to denote any nonenzymatic activity mediated by the acid ceramidase represented by the sequence SEQ ID NO 2, by the mature form, and also by the forms which have or have not undergone glycosylations resulting in molecular weight variants.

This modulating agent may be an agent for activating or inhibiting the gene or protein expression of a polypeptide of the invention.

By way of nonlimiting illustration of the agents for activating the expression of a polypeptide of the invention, mention may be made of compounds for promoting keratinocyte differentiation, such as PPAR-gamma agonists, inhibitors of histone deacetylase, such as butyrate and derivatives thereof, and molecules such as 2-(3,4,5-trimethoxyphenylamino)pyrrolo[2,3-d]pyrimidine and derivatives thereof.

By way of nonlimiting illustration of the agents for inhibiting the expression of a polypeptide of the invention, mention may be made of compounds for decreasing keratinocyte differentiation, such as retinoic acid and isomers thereof, retinol and esters thereof, or vitamin D and derivatives thereof. In embodiments, the modulating agent may be an activator of the gene or protein expression of the polypeptides according to the invention.

A modulating agent suitable for the invention may also be an activator or an inhibitor of the enzymatic activity of acid ceramidase.

By way of nonlimiting illustration of the agents for inhibiting the enzymatic activity, mention may be made of N-oleylethanolamine, certain ceramide analogues such as B13, and D-NMAPPD.

By way of nonlimiting illustration of the agents for activating the enzymatic activity, mention may be made of saposins C and D and certain peptide fragments thereof, or amphiphilic chemical analogues of these natural activators.

In embodiments, the modulating agent may be an activator of the enzymatic activity of the polypeptides according to the invention.

The present invention relates, in addition, to a method for screening for biological or chemical compounds or for physicochemical factors capable of modulating a biological or biochemical activity of a polypeptide according to the invention, comprising at least the steps consisting in:

a) bringing at least one polypeptide in accordance with the invention into contact with at least one chemical or biological test compound, and/or subjecting said polypeptide to said physicochemical factor, under conditions suitable for the manifestation of said biological or biochemical activity of said polypeptide, and

b) determining said biological or biochemical activity of said polypeptide.

According to one embodiment, the biological or biochemical activity of the polypeptide may be compared to a reference value.

A reference value may be obtained by measuring the biological or biochemical activity of the polypeptide in the absence of any biological or chemical test compound or physicochemical test factor.

In the event that this reference value measurement is carried out prior to the use of the biological or chemical test compound or of the physicochemical test factor, the method according to the invention may make it possible, where appropriate, to assess the potential effectiveness of said compound.

This biological or biochemical activity may not be affected by the presence of said compound or, on the other hand, may be inhibited or stimulated.

In the event that an inhibitory effect is noted, the compound tested is capable of being used, for example, as an anti-aging active agent.

A method in accordance with the invention may be carried out on an isolated cell sample, obtained either from a skin biopsy or from cells in culture.

Advantageously, by way of a cell sample suitable for the invention, mention may be made of a keratinocyte sample.

Advantageously, a polypeptide used in a method according to the present invention may be acid ceramidase.

The present invention also relates to a method for screening for biological or chemical compounds capable of modulating the expression of a polypeptide in accordance with the invention, comprising at least the steps consisting in:

a) bringing at least one cell type capable of expressing a nucleic acid sequence encoding said polypeptide in accordance with the invention into contact with at least one chemical or biological test compound, under conditions suitable for the manifestation of the expression of said sequence, and

b) determining the expression of said nucleic acid sequence.

The expression of a nucleic acid sequence can be determined, for example, by means of oligonucleotide probes, by any protocol known to those skilled in the art.

By way of example of methods for detecting a nucleic acid sequence, mention may be made of quantitative (Q-PCR) or nonquantitative polymerase chain reaction (PCR), in the presence or absence of reverse transcriptase (RT-PCR or Q-RT-PCR), Northern blotting, ribonuclease protection assay method, methods with DNA chips, methods with transcriptome chips, methods with oligonucleotide chips, and in situ hybridization methods.

By way of example of agents suitable for the detection of a nucleic acid sequence, such as mRNA, mention may be made of a labeled nucleic acid probe that can hybridize to said sequence.

Such a nucleic acid probe can be readily obtained by any method known to those skilled in the art.

Thus, the nucleic acid sequences in accordance with the invention may be used to prepare sense and/or antisense oligonucleotide primers, which hybridize, under high stringency conditions, to the sequence SEQ ID NO 1 or an analogue thereof.

The expression of a nucleic acid sequence in accordance with the invention can be compared to a reference value obtained, for example, by carrying out a method in accordance with the invention in the absence of test compound.

The expression of a nucleic acid sequence may also be determined, indirectly, by determining the expression of the polypeptide encoded by said sequence, by means of any technique known in the field, such as Western blotting, ELISA, the Bradford or Lowry method, or as indicated hereinafter.

The present invention also relates to a method for screening for biological or chemical compounds, or even for anti-aging active agents, capable of modulating the expression of a polypeptide in accordance with the invention, comprising at least the steps consisting in:

a) bringing at least one cell type capable of expressing a polypeptide in accordance with the invention into contact with at least one chemical or biological test compound, under conditions suitable for the manifestation of the expression of said polypeptide,

b) determining the polypeptide content, and

c) comparing said content determined in step b) to the content of said polypeptide determined in the absence of chemical or biological test compound.

The comparison carried out in step c) can make it possible to deduce a piece of information regarding the suitability of said tested compound for modulating the expression of a polypeptide in accordance with the invention.

A method in accordance with the invention may be carried out on an isolated cell sample.

The determination of a content of polypeptide in accordance with the invention may be carried out by means of any method known to those skilled in the art.

By way of methods for detecting a polypeptide, mention may be made of Western blotting, slot blotting, dot blotting, ELISA (Enzyme Linked Immunosorbent Assay) methods of the singleplex or multiplex type, proteomics or glycomics methods, staining polypeptides in a polyacrylamide gel with a silver-based stain, with Coomassie blue or with SYPRO, immunofluorescence, UV absorption, immunohistochemical methods in conventional, electron or confocal microscopy, FRET (fluorescence resonance energy transfer), TR-FRET (time resolved FRET) methods, FLIM (fluorescence lifetime imaging microscopy) methods, FSPIM (fluorescence spectral imaging microscopy) methods, FRAP (fluorescence recovery after photobleaching) methods, reporter-gene methods, AFM (atomic force microscopy) methods, surface plasmon resonance methods, microcalorimetry methods, flow cytometry methods, biosensor methods, radioimmuno-assay (RIA) methods, isoelectric focusing methods, and enzyme assays, methods using peptide chips, sugar chips, antibody chips, mass spectrometry methods, and SELDI-TOF spectrometry methods (Ciphergen).

The methods in accordance with the invention may be carried out on a sample for example an isolated sample, of epithelium, such as epidermis, obtained from a skin biopsy or from an epithelial cell model, for example an epidermal cell model, or more advantageously from a noninvasive surface removal, such as adhesive tape (stripping tape), of stratum corneum or by simple washing.

A sample of epidermis can be taken by any method known to those skilled in the art.

These methods may be carried out by “stripping” techniques.

These strippings are sticky surfaces applied to the surface of the epidermis, such as Blenderm® from 3M, D′squam (commercial adhesive from CuDERM), cyanoacrylate glue or the varnish stripping method. By virtue of these strippings, the adherent corneocytes and the content of their intercellular spaces can be sampled and subsequently subjected to an extraction which makes it possible to access the protein content.

The taking of a sample suitable for the method may also be carried out more directly by “washing” the skin surface by means, for example, of accessories of the vane turbine type, of the spiral cell type (as described in patent FR 2 667 778) combined with a fluid circuit, or simply by addition/removal of a drop of buffer at the surface of the skin.

By way of indication of other sampling methods suitable for implementing the invention, mention may be made of methods based on scraping the upper part of the stratum corneum by means of a twin blade system. This technique makes it possible to collect squamae which can then be directly analyzed by various techniques in order to determine the mineral, amino acid or lipid contents.

It is understood that the cosmetic or therapeutic compositions considered according to embodiments of the invention may use a physiologically acceptable medium.

For the purpose of the present invention, the term “physiologically acceptable medium” is intended to denote a medium suitable for the application of a composition to an epithelium or a keratin material, such as the skin, the scalp, the lips, the mucous membranes and keratin fibers such as the hair, the nails and body hairs, or where appropriate, by oral or parenteral administration.

For the purpose of the present invention, the term “therapeutic” is intended to denote a composition that can be used in the context of a prophylactic and/or curative treatment, or of a method for evaluating a state of an epithelium, such as the epidermis.

According to another embodiment, a cosmetic or therapeutic composition may also comprise at least one cosmetic and/or therapeutic active agent.

As examples of active agents that can be used in the context of the present invention, mention may be made of cosmetic oils, such as silicone oils, plant oils of the triglyceride type, hydrocarbon-based oils such as Parleam oil and esters of fatty acids and of fatty alcohols.

It may also be possible to use other active agents which make it possible to improve the condition of the skin, such as hydrating or moisturizing active agents or active agents which make it possible to improve the natural lipid barrier, such as ceramides, cholesterol sulfates and/or fatty acids, and mixtures thereof.

It may also be possible to use enzymes which have an activity on the skin, such as proteases, lipases, glucosidases, amidases, cerebrosidases and/or melanases, and mixtures thereof.

As other examples of active agents suitable for implementing the present invention, mention may be made of: analgesic active agents, anti-yeast active agents, antibacterial active agents, antiparasitic active agents, antifungal active agents, antiviral active agents, steroidal anti-inflammatory active agents, anesthetic active agents, antipruritic active agents, keratolytic active agents, free-radical scavenger active agents, antiseborrhoeic active agents, antidandruff active agents, anti-acne active agents, active agents intended for preventing aging of the skin and/or for improving the condition thereof, anti-dermatitis active agents, antiirritant active agents, immunomodulatory active agents, active agents for the treatment of dry skin, antiperspirant active agents, antipsoriatic active agents, active agents for protecting against UV, antihistamine active agents, cicatrizing active agents, self-tanning active agents, antioxidants such as green tea or active fractions thereof, glycerol, laponite, caffeine, aromatic essential oils, colorants, depigmenting active agents, liporegulators, emollient, refreshing, deodorizing, desensitizing, bleaching or nourishing active agents, active agents for reducing skin differentiation and/or proliferation and/or pigmentation, and mixtures thereof.

In general, any composition may be applied to the skin (on any skin region of the body) or to the mucous membranes (buccal, jugal, gingival, genital, conjunctival, etc.).

In a known manner, a cosmetic composition may also contain adjuvants which are customary in the cosmetics field, such as hydrophilic or lipophilic gelling agents, hydrophilic or lipophilic additives, preservatives, antioxidants, solvents, fragrances, fillers, screens, odor absorbers and dyestuffs.

The amounts of the various constituents of the compositions according to the invention are those conventionally used in the fields under consideration.

The amount of chemical or biological compound or of polypeptide, nucleic acid sequence or modulating agent in accordance with the invention contained in a composition, also referred to as “effective amount”, of course depends on the nature of the compound and on the desired effect, and may therefore vary to a large extent.

To give an order of magnitude, a composition may contain a modulating agent in accordance with the invention or a polypeptide in an amount representing from 0.00001% to 50% of the total weight of the composition, such as in an amount representing from 0.001% to 10% of the total weight of the composition, or in an amount representing from 0.1% to 1% of the total weight of the composition.

In embodiments, a composition according to the invention may be, for example, intended for reducing and/or treating conditions that may cause deterioration of the state of an epithelium, such as an epidermis.

A state of an epithelium may be a state linked to a dysfunction of terminal epithelial, such as epidermal, differentiation of keratinocytes.

Such a state may be of chronological origin (i.e. linked to the time elapsed, such as skin aging) and/or a sign of a skin disorder, linked for example to photoaging.

Thus, a composition in accordance with the invention, such as a cosmetic composition, may be for preventing and/or treating signs of epidermal aging.

A composition in accordance with the invention may be for preventing and/or treating thinning of an epidermis and/or a loss of firmness, of elasticity, of density and/or of tonicity of an epidermis and/or the formation of wrinkles and fine lines.

According to another embodiment, a composition, such as a cosmetic composition, may be for preventing and/or treating cutaneous signs of dryness, such as preventing and/or treating dehydration of an epidermis.

A composition in accordance with the invention, such as a therapeutic composition, may be for use in the treatment of a skin disorder such as a skin hydration disorder, for instance xerosis, parakeratosis, hyperkeratosis, ichthyosis, psoriasis, atopic dermatitis, eczema, rosacea, lichen, pruritus, a skin pathology having an inflammatory component or resulting from an impairment of the immune response, desquamation, disruption of melanogenesis or of sebogenesis, alopecia, hirsutism, a cicatrization disorder, or a skin disorder involving secretion phenomena and cell invasion processes, such as in the context of malignant or benign neoplasias.

According to another aspect, the present invention also relates to the use of at least one polypeptide in accordance with the invention or of at least one nucleic acid sequence encoding said polypeptide, as a tool for characterizing, in vitro or ex vivo, a state of an epithelium, such as an epideimis.

By way of example, it is possible to characterize, according to the invention, a state of an epithelium chosen from dryness, chronological aging and photoaging of an epidermis.

Thus, as is specified above, according to another of its aspects, the present invention relates to noninvasive methods for characterizing the surface state of a nonpathological epidermis or else the effectiveness of a cosmetic or therapeutic treatment, directed toward qualitatively or quantitatively characterizing the expression of acid ceramidase, or of a derivative or fragment thereof.

These methods are particularly advantageous since their implementation does not require obligatory recourse to a surgical technique for carrying out such a characterization. An extract of the epidermis can thus be obtained by simple stripping and directly analyzed by a conventional analytical technique, such as the technique described above.

According to one embodiment, a method for characterizing a state of an epithelium, for example an epidermis, comprises at least the steps consisting in:

a) determining, in a sample of said epithelium, the content of polypeptide in accordance with the invention, or of a nucleic acid sequence encoding said polypeptide, and

b) comparing said content determined in step a) to a reference value.

Advantageously, a method of the invention is noninvasive.

A method of the invention is advantageously carried out on an isolated sample.

According to one embodiment, a method according to the invention may be carried out on a sample of epithelium, such as an epidermis, taken from an individual.

A method according to the invention may also be carried out on a sample of epithelium, such as an epidermis, taken from an epithelial cell model, such as an epidermal cell model, or from a reconstructed isolated skin in order to qualify the state thereof.

A sample of epithelium may be taken by any method known to those skilled in the art.

A method according to the invention may be carried out in vivo, in vitro or ex vivo.

A reference value may, for example, be a content of polypeptide or of nucleic acid sequence determined on a sample of epidermis taken from an epithelium, such as from normal skin, i.e. skin that is satisfactory from a physiological point of view, like, for example, young skin.

A reference value may be measured in parallel with or following the determination of said content of a polypeptide or of a nucleic acid sequence.

A comparison of a determined content with a reference value may make it possible to evaluate a deviation relative to this value.

The analysis of the intensity and/or of the nature of this deviation (negative or positive) may be informative with regard to the state of the epidermis.

The characterization of a state of an epidermis may be indicative of a possible skin disorder which may be corrected by the use of compounds capable of modulating the expression of a polypeptide of the invention.

According to one embodiment, a method according to the invention may be implemented in a method for the in vivo, in vitro or ex vivo diagnosis of aging of an epithelium, such as the epidermis, in an individual.

A polypeptide suitable for carrying out the method according to the invention may advantageously be acid ceramidase.

The determination of the content of polypeptide in accordance with the invention or of nucleic acids in accordance with the invention in a sample of epidermis may be carried out by any protocol known to those skilled in the art.

By way of methods of detecting a polypeptide, mention may be made of those mentioned above.

Thus, it is possible to envision detecting the presence of a polypeptide in accordance with the invention by means of an antibody, where appropriate in a labeled form.

An antibody that can be used as a tool for evaluating a state of an epidermis can be obtained by any method known to those skilled in the art, as described in “Antibodies: A Laboratory Manual”, Cold Spring Harbor Laboratory Press, Cold Spring Harbor, N.Y. (1990). Advantageously, the antibodies used may be recombinant antibodies such as those developed by the company Antibodies-by-design.

A nucleic acid sequence suitable for implementing a method according to the invention may advantageously be a nucleic acid sequence encoding acid ceramidase, for example of the mRNA type.

By way of example of methods for detecting nucleic acids in accordance with the invention, mention may be made of the methods mentioned above.

The present invention also relates to a nontherapeutic method for demonstrating an effect of a treatment capable of causing regression of an epithelial disorder, such as a skin or scalp disorder, in an individual, comprising at least the steps consisting in:

a) carrying out, before the treatment, at least a first determination, in a first sample of an epithelium taken from said individual, of a biological or biochemical activity and/or of the expression of a polypeptide in accordance with the invention, or of the expression of a nucleic acid sequence encoding said polypeptide,

b) carrying out, after the treatment, at least a second determination, in a second sample of an epithelium taken from said individual, of said biological or biochemical activity and/or of said expression of said polypeptide or of said expression of said nucleic acid sequence, determined in step a), and

c) comparing the first and second determinations in order to, for example, deduce therefrom information relating to at least one effect of the treatment.

Such a treatment may, for example, be a cosmetic treatment.

In embodiments, the treatment of which the effect is to be evaluated may be a treatment for relieving or reducing the signs of skin aging or a scalp disorder.

The biological or biochemical activity and the expression of a polypeptide may be determined as indicated above.

According to another aspect, the present invention relates to a method for the cosmetic treatment of the signs of skin aging, comprising at leastone step consisting in applying at least one cosmetic composition in accordance with the invention to at least one part of the skin, mucous membranes and/or keratin fibers.

According to another aspect, the present invention relates to the use of an effective amount of at least one polypeptide in accordance with the invention or of at least one agent for modulating the expression of said polypeptide, for the preparation of and/or for improving a pluristratified cell model, such as of epidermal or mucosal type, and, for example, a reconstructed skin model.

For the purpose of the invention, the term “reconstructed skin model” is intended to denote a model in which various cell types are combined, such as the natural constituents of the skin, like for example keratinocytes, fibroblasts, Langerhans cells and melanocytes.

The cells of the fibroblast type may or may not be irradiated.

Such models and the preparation thereof are known to those skilled in the art.

For the purpose of the present invention, “a” should be understood, unless otherwise indicated, in the sense of “at least one”.

The examples presented below are given by way of nonlimiting illustration of the invention.

### EXAMPLE I

Analysis of Samples Taken By Varnish Stripping on Various Skin Regions of an Individual

The analyses are carried out using varnish strippings performed on the legs.

The individuals suitable for the study are put into 4 groups.

The AS group corresponds to group 1: dry menopausal individuals, n=15.

The AN group corresponds to group 2: normal menopausal individuals, n=13.

The JS group corresponds to group 3: dry young individuals, n=16.

The JN group corresponds to group 4: normal young individuals, n=14.

1: Preparation of Acetone Powders

Two varnish strippings (B. Mehul et al., J Biol Chem 2000, Apr. 28; 275(17): 12841-7) of 10 cm² are placed into 20 ml of acetone. The corneocytes become detached. The mixture is filtered through a 40 μm nylon membrane. Three successive rinses are carried out with the same volume of acetone. The suspension is finally filtered on a vacuum pump. Acetone powders of stratum corneum are obtained in dry form.

2: Sample Extraction

An extraction is carried out under denaturing conditions. To do this, a prewash is carried out with a volume (100 μl) of PBS buffer (phosphate buffered saline) +0.1% Triton X100, which is added per mg of acetone powder. The mixture is ground in a Potter and centrifuged. The corneocyte pellet is recovered. It is extracted with the same volume (100 μl/mg) of Laemmli buffer containing 0.0625 mM Tris, pH 6.8, 200 mM DTT, 2% SDS and 10% glycerol. The mixture is heated at boiling temperature for 10 minutes, and is then ground and centrifuged for 10 minutes at 10 000 g. The supernatant is recovered. A protein assay is carried out according to the Bradford technique with the Bradford reagent (Bio-Rad protein assay). The samples are adjusted to 1 mg/ml.

3: Protein Analysis by Western Blotting

The proteins are separated by SDS-PAGE electrophoresis. After semi-dry blotting onto a PVDF membrane (Immobilon-P, Millipore) according to a standard protocol, the proteins are incubated with the anti-acid ceramidase primary antibody (BD612303) overnight at 4° C. The second incubation is then carried out with the secondary antibody (mouse IgG HRP conjugate) (Bio-Rad) directed against the primary antibody for 1 h 30 at ambient temperature. The presence of acid ceramidase on the membrane is revealed by immunodetection using the ECL Plus kit (Amersham). The membrane is then stained with amido black in order to detect the total proteins present on the membrane. The image is acquired with FluorSmax (Biorad) and the bands are quantified using the Quantity-one software (Biorad).

4: Results

The results are expressed as delta cnt*mm² of the protein of interest/delta cnt*mm² of total proteins.

### Methodology:

2-way (age and type) analysis of variance of skin taking into account the interaction of these two factors+1-way (group) analysis of variance and Tukey's multiple comparison test. Since the normality and homoscedasticity conditions were not verified, the analysis was carried out after logarithmic transformation.

The statistical analysis was carried out with the SAS version 8.2 and SPSS version 12 software packages.

All the tests were carried out at the 5% two-sided threshold.

The table below gives the mean results and also their standard errors of the mean (sem).

| Group | Acid ceramidase | sem acid ceramidase |
|---|---|---|
| AS | 1518 | 267 |
| AN | 1378 | 227 |
| JS | 1875 | 183 |
| JN | 2006 | 277 |

A significant decrease is noted in the expression of acid ceramidase during skin aging (p=0.047).

### Sequence Listing

| SEQ ID NO 1 | |
|---|---|
| 1 | ggcacgaggc tagagcgatg ccgggccgga gttgcgtcgc cttagtcctc ctggctgccg |
| 61 | ccgtcagctg tgccgtcgcg cagcacgcgc cgccgtggac agaggactgc agaaaatcaa |
| 121 | cctatcctcc ttcaggacca acgtacagag gtgcagttcc atggtacacc ataaatcttg |
| 181 | acttaccacc ctacaaaaga tggcatgaat tgatgcttga caaggcacca atgctaaagg |
| 241 | ttatagtgaa ttctctgaag aatatgataa atacattcgt gccaagtgga aaagttatgc |
| 301 | aggtggtgga tgaaaaattg cctggcctac ttggcaactt tcctggccct tttgaagagg |
| 361 | aaatgaaggg tattgccgct gttactgata tacctttagg agagattatt tcattcaata |
| 421 | ttttttatga attatttacc atttgtactt caatagtagc agaagacaaa aaaggtcatc |
| 481 | taatacatgg gagaaacatg gattttggag tatttcttgg gtggaacata aataatgata |
| 541 | cctgggtcat aactgagcaa ctaaaacctt taacagtgaa tttggatttc caaagaaaca |
| 601 | acaaaactgt cttcaaggct tcaagctttg ctggctatgt gggcatgtta acaggattca |
| 661 | aaccaggact gttcagtctt acactgaatg aacgtttcag tataaatggt ggttatctgg |
| 721 | gtattctaga atggattctg ggaaagaaag atgccatgtg gatagggttc ctcactagaa |
| 781 | cagttctgga aaatagcaca agttatgaag aagccaagaa tttattgacc aagaccaaga |
| 841 | tattggcccc agcctacttt atcctgggag gcaaccagtc tggggaaggt tgtgtgatta |
| 901 | cacgagacag aaaggaatca ttggatgtat atgaactcga tgctaagcag ggtagatggt |
| 961 | atgtggtaca aacaaattat gaccgttgga aacatccctt cttccttgat gatcgcagaa |
| 1021 | cgcctgcaaa gatgtgtctg aaccgcacca gccaagagaa tatctcattt gaaaccatgt |
| 1081 | atgatgtcct gtcaacaaaa cctgtcctca acaagctgac cgtatacaca accttgatag |
| 1141 | atgttaccaa aggtcaattc gaaacttacc tgcgggactg ccctgaccct tgtataggtt |
| 1201 | ggtgagcaca cgtctggcct acagaatgcg gcctctgaga catgaagaca ccatctccat |
| 1261 | gtgaccgaac actgcagctg tctgaccttc caaagactaa gactcgcggc aggttctctt |
| 1321 | tgagtcaata gcttgtcttc gtccatctgt tgacaaatga cagatctttt tttttttccc |
| 1381 | cctatcagtt gatttttctt atttacagat aacttcttta ggggaagtaa aaaggtcatc |
| 1441 | tagaattcac tgagttttgt ttcactttga catttgggga tctggtgggc agtcgaacca |
| 1501 | tggtgaactc cacctccgtg gaataaatgg agattcagcg tgggtgttga atccagcacg |
| 1561 | tctgtgtgag taacgggaca gtaaacactc cacattcttc agtttttcac ttctacctac |
| 1621 | atatttgtat gtttttctgt ataacagcct tttccttctg gttctaactg ctgttaaaat |
| 1681 | taatatatca ttatctttgc tgttattgac agcgatatta ttttattaca tatcattaga |
| 1741 | gggatgagac agacattcac ctgtatattt cttttaatgg gcacaaaatg ggcccttgcc |
| 1801 | tctaaatagc actttttggg gttcaagaag taatcagtat gcaaagcaat cttttataca |
| 1861 | ataattgaag tgttcccttt ttcataatta ctctacttcc cagtaaccct aaggaagttg |
| 1921 | ctaacttaaa aaactgcatc ccacgttctg ttaatttagt aaataaacaa gtcaaagact |
| 1981 | tgtggaaaat aggaagtgaa cccatatttt aaattctcat aagtagcatt gatgtaataa |
| 2041 | acaggttttt agtttgttct tcagattgat agggagtttt aaagaaattt tagtagttac |
| 2101 | taaaattatg ttactgtatt tttcagaaat caaactgctt atgaaaagta ctaatagaac |
| 2161 | ttgttaacct ttctaacctt cacgattaac tgtgaaatgt acgtcatttg tgcaagaccg |
| 2221 | tttgtccact tcattttgta taatcacagt tgtgttcctg acactcaata aacagtcact |
| 2281 | ggaaagagtg ccagtcagca gtcatgcacg ctgataaaaa aaaaaaaaaa aaa |
| SEQ ID NO 2 | |
| 1 | MPGRSCVALV LLAAAVSCAV AQHAPPWTED CRKSTYPPSG PTYRGAVPWY TINLDLPPYK |
| 61 | RWHELMLDKA PMLKVIVNSL KNMINTFVPS GKVMQVVDEK LPGLLGNFPG PFEEEMKGIA |
| 121 | AVTDIPLGEI ISFNIFYELF TICTSIVAED KKGHLIHGRN MDFGVFLGWN INNDTWVITE |
| 181 | QLKPLTVNLD FQRNNKTVFK ASSFAGYVGM LTGFKPGLFS LTLNERFSIN GGYLGILEWI |
| 241 | LGKKDAMWIG FLTRTVLENS TSYEEAKNLL TKTKILAPAY FILGGNQSGE GCVITRDRKE |
| 301 | SLDVYELDAK QGRWYVVQTN YDRWKHPFFL DDRRTPAKMC LNRTSQENIS FETMYDVLST |
| 361 | KPVLNKLTVY TTLIDVTKGQ FETYLRDCPD PCIGW |

SEQ ID NO 3
ASSFAGYVGMLTGFKPGLFSLTLNER
SEQ ID NO 4
ESLDVYELDAK
SEQ ID NO 5
ILAPAYFILGGNQSGEGCVITR
SEQ ID NO 6
KDAMWIGFLTR
SEQ ID NO 7
LPGLLGNFPGPFEEEMK
SEQ ID NO 8
LTVYTTLIDVTKGQFETYLR
SEQ ID NO 9
STYPPSGPTYRGAVPWYTINLDLPPYK
SEQ ID NO 10
TSQENISFETMYDVLSTKPVLNKLTVYTTLIDVTKGQFETYLR
SEQ ID NO 11
VIVNSLK
SEQ ID NO 12
WHELMLDK
SEQ ID NO 13
WYVVQTNYDRWKHPFFLDDR

1. A method for preventing and/or treating the signs of skin aging comprising administering as an agent into an individual in need thereof an effective amount of:
at least one polypeptide having an amino acid sequence encoded by a nucleic acid sequence represented entirely or partly by a sequence represented by SEQ ID NO: 1, an analogue thereof or a fragment thereof,
at least one nucleic sequence encoding said polypeptide; or
at least one agent for modulating the expression or the biological or biochemical activity of said polypeptide.

2. A therapeutic method for preventing and/or treating the signs of skin aging comprising administering into an individual in need thereof a therapeutic composition comprising an effective amount of:
at least one polypeptide having an amino acid sequence encoded by a nucleic acid sequence represented entirely or partly by a sequence represented by SEQ ID NO: 1, an analogue thereof or a fragment thereof,
at least one nucleic acid sequence encoding said polypeptide; or
at least one agent for modulating the expression or the biological or biochemical activity of said polypeptide.

3. The method as claimed in claim 1, wherein said polypeptide has an amino acid sequence represented entirely or partly by a sequence represented by SEQ ID NO: 2, an analogue thereof or a fragment thereof.

4. The method as claimed in claim 1, wherein said polypeptide has an amino acid sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 and SEQ ID NO: 13.

5. The method as claimed in claim 1, wherein the modulating agent is an activator of the gene or protein expression of said polypeptide.

6. The method as claimed in claim 5, wherein said activator is selected from the group consisting of PPAR-gamma agonists, inhibitors of histone deacetylase, and 2-(3,4,5-trimethoxyphenylamino)pyrrolo-[2,3-d]pyrimidine and derivatives thereof.

7. The method as claimed in claim 1, wherein the modulating agent is an activator of the enzymatic activity of said polypeptide.

8. The method as claimed in claim 1, wherein said agent is for preventing and/or treating thinning of an epidermis and/or a loss of firmness, of elasticity, of density and/or of tonicity of an epidermis and/or the formation of wrinkles and fine lines.

9. The method as claimed in claim 1, wherein said agent is for preventing and/or treating cutaneous signs of dryness.

10. The method for characterizing, in vitro or ex vivo, a state of an epithelium comprising a step of determining a content of at least one polypeptide having an amino acid sequence encoded by a nucleic acid sequence represented entirely or partly by a sequence represented by SEQ ID NO: 1, an analogue thereof or a fragment thereof, or of at least one nucleic acid sequence encoding said polypeptide in an epidermis sample.

11. The method as claimed in claim 10, wherein the state of the epithelium is selected from the group consisting of dryness, chronological aging and photoaging of an epidermis.

12. A method for characterizing a state of an epithelium, comprising:
a) determining, in a sample of said epithelium, a content of a polypeptide having an amino acid sequence encoded by a nucleic acid sequence represented entirely or partly by a sequence represented by SEQ ID NO: 1, an analogue thereof or a fragment thereof, or a nucleic acid sequence encoding said polypeptide, and
b) comparing said content determined in step a) to a reference value.

13. The method as claimed in claim 12, wherein the method is noninvasive.

14. A method for screening for anti-aging active agents, comprising:
a) bringing at least one cell type capable of expressing a polypeptide having an amino acid sequence encoded by a nucleic acid sequence represented entirely or partly by a sequence represented by SEQ ID NO: 1, an analogue thereof or a fragment thereof, into contact with at least one chemical or biological test compound, under conditions suitable for manifestation of the expression of said polypeptide, and
b) determining the content of said polypeptide.

15. A cosmetic method for characterizing the effectiveness of a cosmetic or therapeutic treatment aiming to compensate for the signs of skin aging, comprising at least the qualitative or quantitative characterization of the expression and/or of the biological or biochemical activity of a polypeptide having an amino acid sequence encoded by a nucleic acid sequence represented entirely or partly by a sequence represented by SEQ ID NO: 1, an analogue thereof or a fragment thereof.

16. The method for preparing and/or improving a pluristratified cell model comprising adding to said pluristratified call model an effective amount of at least one polypeptide having an amino acid sequence encoded by a nucleic acid sequence represented entirely or partly by a sequence represented by SEQ ID NO: 1, an analogue thereof or a fragment thereof, or at least one agent for modulating the expression of said polypeptide.

17. The method as claimed in claim 2, wherein said polypeptide has an amino acid sequence represented entirely or partly by a sequence represented by SEQ ID NO: 2, an analogue thereof or a fragment thereof.

18. The method as claimed in claim 2, wherein said polypeptide has an amino acid sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 and SEQ ID NO: 13.

19. The method as claimed in claim 2, wherein the modulating agent is an activator of the gene or protein expression of said polypeptide.

20. The method as claimed in claim 19, wherein said activator is selected from the group consisting of PPAR-gamma agonists, inhibitors of histone deacetylase, and 2-(3,4,5-trimethoxyphenylamino)pyrrolo[2,3-d]pyrimidine and derivatives thereof.

21. The method as claimed in claim 2, wherein the modulating agent is an activator of the enzymatic activity of said polypeptide.

22. The method as claimed in claim 2, wherein said agent is for preventing and/or treating thinning of an epidermis, and/or a loss of firmness, elasticity, density and/or tonicity of an epidermis, and/or the formation of wrinkles and fine lines.

23. The method as claimed in claim 2, wherein said agent is for preventing and/or treating cutaneous signs of dryness.
